(19) United States
(12) Patent Application Publication
(10) Pub. No.: US 2025/0017500 A1
(43) Pub. Date: Jan. 16, 2025

(54) SOFT BOOT PULSE OXIMETRY SENSOR

(71) Applicant: MASIMO CORPORATION, Irvine, CA (US)

(72) Inventors: Charles D. O'Neil, Mission Viejo, CA (US); Sujin Hwang, Irvine, CA (US); John Schmidt, Lake Forest, CA (US)

(21) Appl. No.: 18/901,533

(22) Filed: Sep. 30, 2024

Related U.S. Application Data
(63) Continuation of application No. 18/166,172, filed on Feb. 8, 2023, now Pat. No. 12,127,834.
Application No. 18/166,172 is a continuation of application No. 16/798,043, filed on Feb. 21, 2020, now Pat. No. 11,602,289.
Application No. 16/798,043 is a continuation of application No. 15/017,217, filed on Feb. 5, 2016, now Pat. No. 10,568,553.
(60) Provisional application No. 62/113,279, filed on Feb. 6, 2015.
(60) Provisional application No. 62/118,668, filed on Feb. 20, 2015.

(51) Int. Cl. A61B 5/1455; A61B 5/00

(52) U.S. Cl. CPC A61B 5/14552; A61B 5/6826; A61B 2560/0443

(57) ABSTRACT

A reusable sensor is disclosed for producing a signal indicative of at least one physiological parameter of tissue. The sensor can include a sensor housing that has a distal opening, a wire lumen, and a proximal opening. The distal opening can include a lumen extending through the body of the sensor housing and the wire lumen can be located on the outside of the sensor housing. The sensor can also include a first component located on a top surface of the sensor housing and along the pathway of the wire lumen. The sensor can also include a second component located on the bottom surface of the sensor housing opposite of the first component. The second component can also be located along the pathway of the wire lumen. The sensor can also include a wire coaxially disposed within the wire lumen and connecting the first component and second component.

### INCORPORATION BY REFERENCE TO ANY PRIORITY APPLICATIONS

This application is a continuation of U.S. patent application Ser. No. 18/166,172, filed Feb. 8, 2023, which is a continuation of U.S. patent application Ser. No. 16/798,043, filed Feb. 21, 2020, which is a continuation of U.S. patent application Ser. No. 15/017,217, filed Feb. 5, 2016, which claims the priority benefit under 35 U.S.C. § 1 19 (e) of U.S. Provisional Application No. 62/113,279, filed Feb. 6, 2015, and U.S. Provisional Application No. 62/118,668, filed Feb. 20, 2015, the entire contents of which are hereby incorporated by reference and should be considered a part of this specification. Any and all applications for which a foreign or domestic priority claim is identified in the Application Data Sheet as filed with the present application are hereby incorporated by reference under 37 CFR 1.57.

### FIELD OF THE DISCLOSURE

The present disclosure relates to sensors. More specifically, the present disclosure relates to reusable medical sensors.

### BACKGROUND

Energy is often transmitted through or reflected from a medium to determine characteristics of the medium. For example, in the medical field, instead of extracting material from an individual's body for testing, light or sound energy may be caused to be incident on the individual's body and transmitted (or reflected) energy may be measured to determine information about the material through which the energy has passed. This type of non-invasive measurement is more comfortable for the individual and can be performed more quickly.

Non-invasive physiological monitoring of bodily function is often required. For example, during surgery, blood pressure and the body's available supply of oxygen, or the blood oxygen saturation, are often monitored. Measurements such as these are often performed with non-invasive techniques where assessments are made by measuring the ratio of incident to transmitted (or reflected) light through a portion of the body, for example a digit such as a finger, or an earlobe, or a forehead.

### SUMMARY OF THE DISCLOSURE

The present disclosure relates to a sensor that Is responsive to signals received from the individual. The sensor can be attached to instruments that are responsive to signals from the sensors or the cables from the instrument. In some aspects of the present disclosure, the sensor housing is sufficiently durable for use in non-traditional hospital settings. In some embodiments, the sensor has an opening that allows a light to shine through the surface of the sensor. This can provide a visual indicator to allow for proper sensor placement in low light conditions. In one embodiment, the configuration of the wiring and components inside the sensor housing may be positioned to allow the manipulation of the sensor housing (e.g. for cleaning) while maintaining the integrity of the wiring within the sensor housing. In another embodiment, the sensor housing can provide for the physiological monitoring of bodily function in a non-traditional hospital setting. In one example, the sensor housing can have protruding components to ensure a reliable reading from the individual's fingertip. As well, the sensor housing can have an indicator to notify the individual that the sensor is properly reading the individual's bodily functions.

The sensor provides a comfortable and adjustable fit that can accommodate a patient with any sized finger or fingernail. As well, the structure of the sensor housing both secures the patient's finger to ensure a proper reading by the sensor, but also secures the sensor housing against the patient's finger to prevent improper sensor measurements as a result of the sensor housing bumping against external surfaces. In some embodiments, the sensor housing can include an opening at the proximal end of the sensor housing to allow the fingernail of a finger to extend through the sensor housing. In some embodiments, the sensor housing includes a cable that is located across the top surface of the sensor housing and extends past the proximal end of the sensor housing to provide sufficient room for the fingernail of a finger to be housed in the sensor housing.

The present disclosure provides a durable and reusable optical probe which is suitably constructed to provide low-noise signals to be output to a signal processor in order to determine the characteristics of the medium.

### BRIEF DESCRIPTION OF THE DRAWINGS

FIG. 1A illustrates a top perspective view of one embodiment of the sensor.

FIG. 1B illustrates a circuit diagram of one embodiment of the sensor.

FIG. 2 illustrates a bottom perspective view of one embodiment of the sensor as illustrated in FIG. 1.

FIG. 3 illustrates a back view of one embodiment of the sensor as illustrated in FIG. 1.

FIG. 4 illustrates a front view of one embodiment of the sensor as illustrated in FIG. 1.

FIGS. 5-6 illustrate two side views of one embodiment of the sensor as illustrated in FIG. 1.

FIG. 7 illustrates a top view of one embodiment of the sensor as illustrated in FIG. 1.

FIG. 8 illustrates a bottom view of one embodiment of the sensor as illustrated in FIG. 1.

FIG. 9 illustrates a side cross-sectional view of one embodiment of the sensor as illustrated in FIG. 1.

FIG. 10 illustrates a front cross-sectional view of one embodiment of the sensor as illustrated in FIG. 1.

FIGS. 11A-11F illustrate various perspectives of an alternative embodiment of the sensor.

FIGS. 12A-12C illustrate various perspectives of the embodiment of the sensor shown in FIGS. 11A-11F while in use.

### DETAILED DESCRIPTION

The present disclosure relates to a sensor that is responsive to signals received from the individual. The sensor can be attached to instruments that are responsive to signals from the sensors or the cables from the instrument. In particular, the present disclosure relates to durable and reusable sensors that can be used in challenging environments where traditional reusable style sensors can be damaged. Such sensors are designed for individuals such as firefighters, emergency medical technicians (“EMTs”), and other emergency workers that will have a need for physiological monitoring of bodily functions.

As will be discussed below, the presently disclosed sensor can include a number of features that provide the sensor with increased durability and allow the sensor to be cleaned and reused. The sensor can also have additional features that assist in monitoring of bodily functions in non-controlled environments. In one example, the sensor can be configured with an external structure to prevent the sensor from catching onto outside surfaces and disrupting sensor measurements. In other examples, the sensor can additionally have features that provide visual indications to the individual as to whether the sensor is properly functioning.

As used in the specification, the terms “proximal” and “distal” should be understood as being relative to the location on the sensor where the sensor is monitoring an individual's bodily function (e.g. fingertip). The term “distal” means the portion of the sensor where the finger is first inserted into the sensor housing. The term proximal means the portion of the sensor that is closest the fingertip of the patient when the sensor is placed on the finger of the individual.

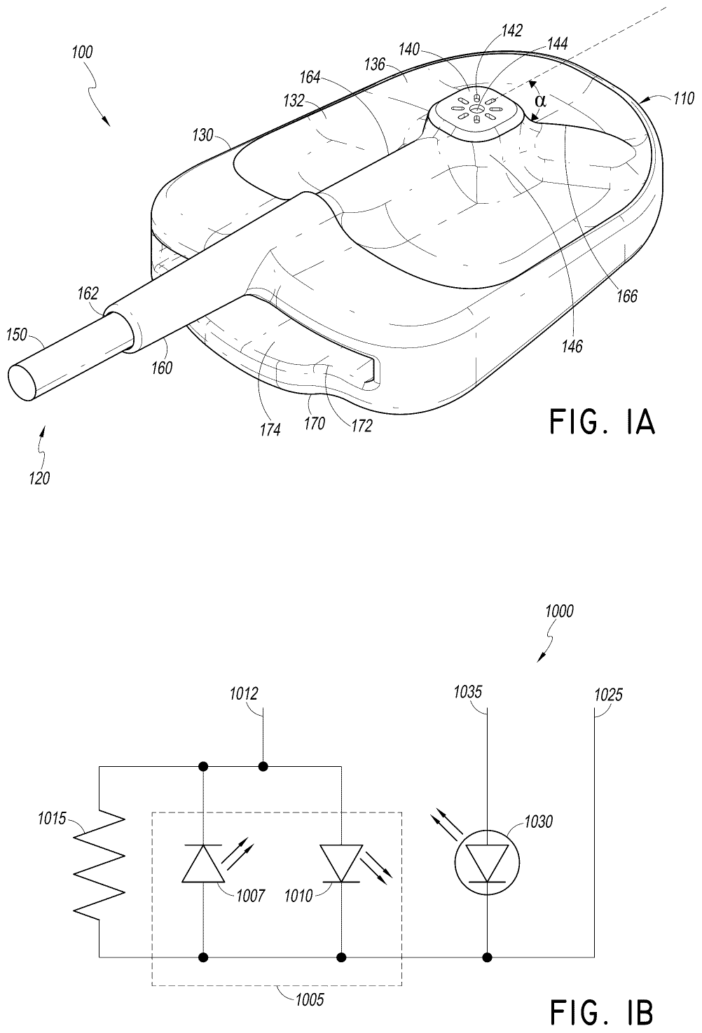

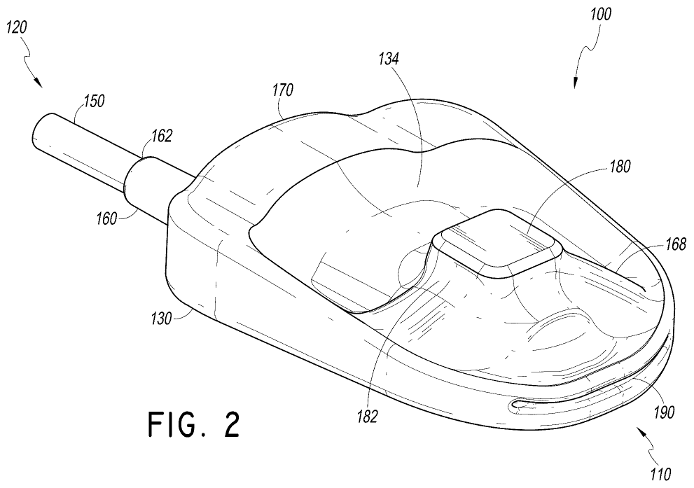

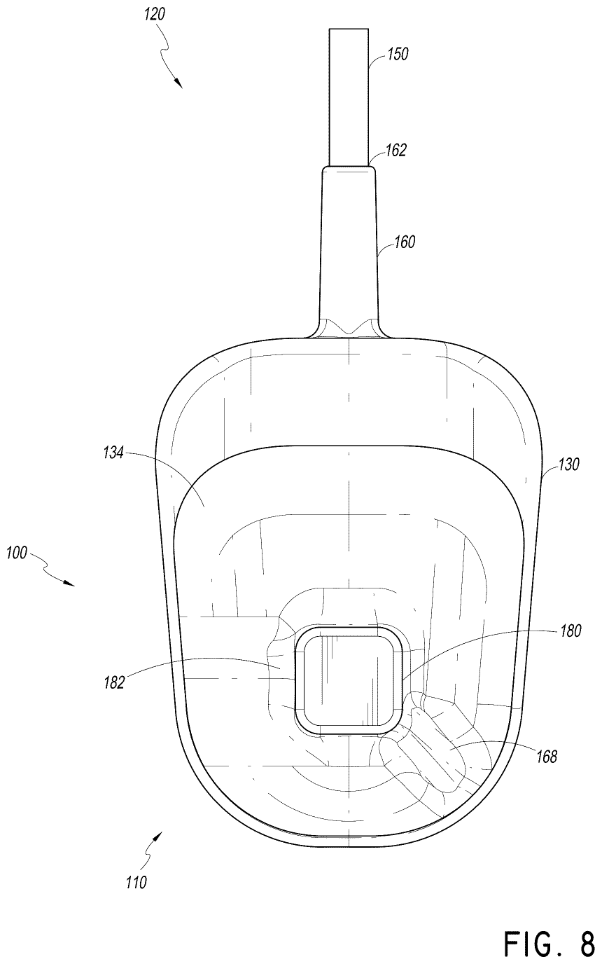

FIGS. 1-10 illustrates a plurality of views of sensor 100. The sensor 100 includes a sensor housing 130, wiring 150, an emitter 146, and a hard shell box 180. The sensor 100 has a proximal end 110 and a distal end 120. As will be discussed in more detail below, the wiring 150 enters the sensor housing 130 from the distal end 120 end of the sensor 100. As well, the sensor housing 130 can also have an opening 1270 on the distal end 120 that allows an individual's finger to fit into the sensor housing 130. On the proximal end 110 of the sensor housing 13 0, the sensor housing 13 0 includes the emitter 146 on the top of the distal end 120 of the sensor housing 13 0 and a detector 182 on the bottom of the distal end 120 of the sensor housing 130. The emitter 146 and detector 182 can interact to read predetermined bodily functions from the individual's finger that are placed in the sensor housing 130. The wiring 150 bends at an angle about the proximal end 110 of the sensor housing 130 to connect the emitter 146 and detector 182. As will be discussed in more detail below, in one embodiment, the wiring 150 can bend at the distal end 120 of the sensor housing 130 to allow the sensor housing 130 to be flipped inside out without damaging the wiring 150.

FIG. 1B illustrates an oximeter sensor circuit 1000 of the sensor 100 described above. As can be seen, the oximeter sensor circuit 1000 includes an emitter 1005 comprising a first LED 1007 and a second LED 1010. The oximeter sensor circuit 1000 further includes an information element comprising a resistor 1015. The first LED 1007, the second LED 1010, and the resistor 1015 are connected in parallel. The parallel connection has a common input electrical connection 1012 and a common return 1025. The oximeter sensor circuit 1000 also includes a detector 1030 having an input electrical connection 1035 connected to one end and having the common return 1025 connected to the other end. In some embodiments, the detector 1030 is a photodetector.

As mentioned, the resistor 1015 is provided as an information element that can be read by an attached oximeter. In order to read the resistor 1015, the oximeter drives the oximeter sensor circuit 1000 at a level where the emitter 1005 draws effectively insignificant current. Because the emitter 1005 becomes active only if driven at a voltage above a threshold level, at this low level, significantly all of the current through the common input electrical connection 1012 flows through the resistor 1015. By reducing the drive voltage across the input electrical connection 1012 and common return 1025 to a low enough level to not activate the emitter 1005, the emitter 1005 is effectively removed from the oximeter sensor circuit 1000. Thus, the oximeter can determine the value of the resistor 1015.

The value of the resistor 1015 can be preselected to indicate, for example, the type of sensor (e.g., adult, pediatric, or neonatal), the operating wavelength, or other parameters about the sensor. The resistor 1015 may also be utilized for security and quality control purposes. For example, the resistor 1015 may be used to ensure that the oximeter sensor circuit 1000 is configured properly for a given oximeter. For instance, the resistor 1015 may be utilized to indicate that the oximeter sensor circuit 1000 is from an authorized supplier.

An information element other than the resistor 1015 may also be utilized. The information element need not be a passive device. Coding information may also be provided through an active circuit, such as a transistor network, memory chip (e.g. EEPROM), or other identification device.

Furthermore, it will be understood by a skilled artisan that a number of different circuit configurations can be implemented that allow the oximeter sensor circuit 1000 to include an information element. For example, the emitter 1005 and the information element may each have individual electrical connections.

As mentioned above, the resistor 1015 is preselected such that at low drive voltages, it is the only circuit element sensed by the oximeter. On the other hand, the resistor 1015 can also be preselected and of a sufficiently high value that when the drive voltage rises to a level sufficient to drive the emitter 100 5, the resistor 1015 is effectively removed from the oximeter sensor circuit 1000. Thus the resistor 1015 does not affect normal operations of the emitter 1005. In summary, an information element may form an integral part of the oximeter sensor circuit 1000 by providing valuable information to the attached oximeter.

The circuit diagram of FIG. 1B is provided as an example of a configuration of the circuitry of the sensor 100 within the sensor housing 130 and is not intended to be limiting. In other embodiments, the oximeter sensor circuit 1000 can include a plurality of emitters 1005 and detectors 1030. For example, instead of/in addition to the resistor 1015, the oximeter sensor circuit 1000 can include additional resistor 1015 or other elements in order to serve as an information element.

As can be seen in FIGS. 1-10, the sensor 100 includes a sensor housing 130. In one embodiment, the sensor housing 130 can be made of a, or elastomeric material, such as, for example, Dynaflex™ thermoplastic elastomer. The rubber or elastomeric material of the sensor housing 13 0 protects the components of the sensor 100 and is flexible so as to resist tearing, cracking or crushing. The material design of the sensor housing 130 also allows the sensor housing 130 to be flipped inside out and for easy cleaning of the finger-contact surfaces. Further, as will be discussed below, while the sensor housing 130 is flexible, it is nevertheless sufficiently rigid so as to conform to the individual's finger and to hold the components of the sensor 100 near the portion of the individual's finger to obtain measurements. In other embodiments, the sensor housing 130 can also be made of silicone or other related material which provides the sensor housing 13 0 with similar properties.

The sensor housing 130 has a top compressed portion 132, a bottom compressed portion 134, a slit 190, a lip 170, and a wiring lumen 160 that partially extends from the sensor housing 130 and houses the wiring 150 through the length of the sensor housing 130. Further, the sensor housing 130 can retain the components of the sensor 100-here, the emitter 146 and detector 182 on the top and bottom surfaces of the distal end 120 of the sensor housing 13 0. The housing 13 0 surrounds the finger. In an embodiment, when the finger is inserted into the sensor, the sensor encloses the finger to block ambient light from entering the sensor housing.

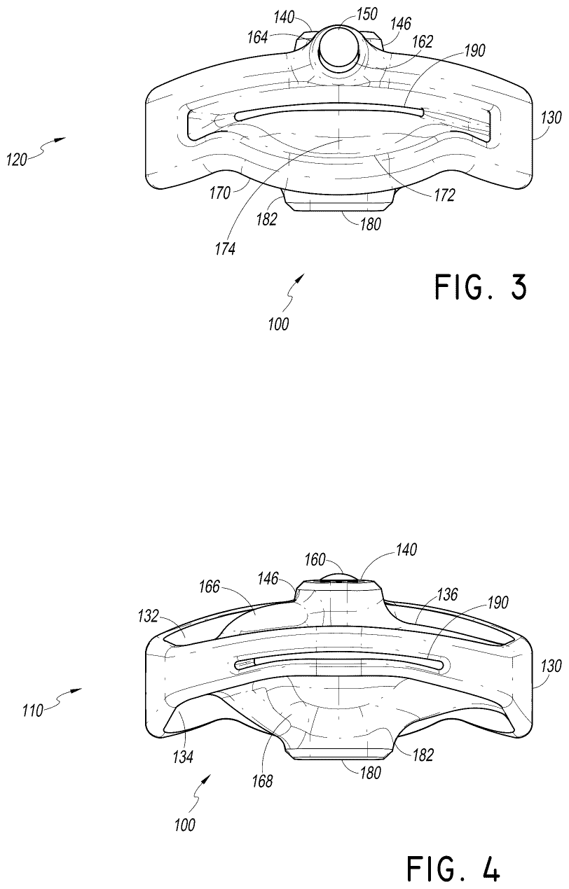

As can be seen m FIGS. 1-2, the sensor housing 130 has a top compressed portion 132 and a bottom compressed portion 134 such that the proximal end 110 of the sensor housing 130 has a more compressed, squeezed or flattened configuration than the distal end 120 of the sensor housing 130. As will be discussed in more detail, the emitter 146 and the hard shell box 180 are contained on the sensor housing 130 on the top compressed portion 132 and bottom compressed portion 134 respectively so as to bring the components of the sensor 100 closer to the finger to improve the physiological reading.

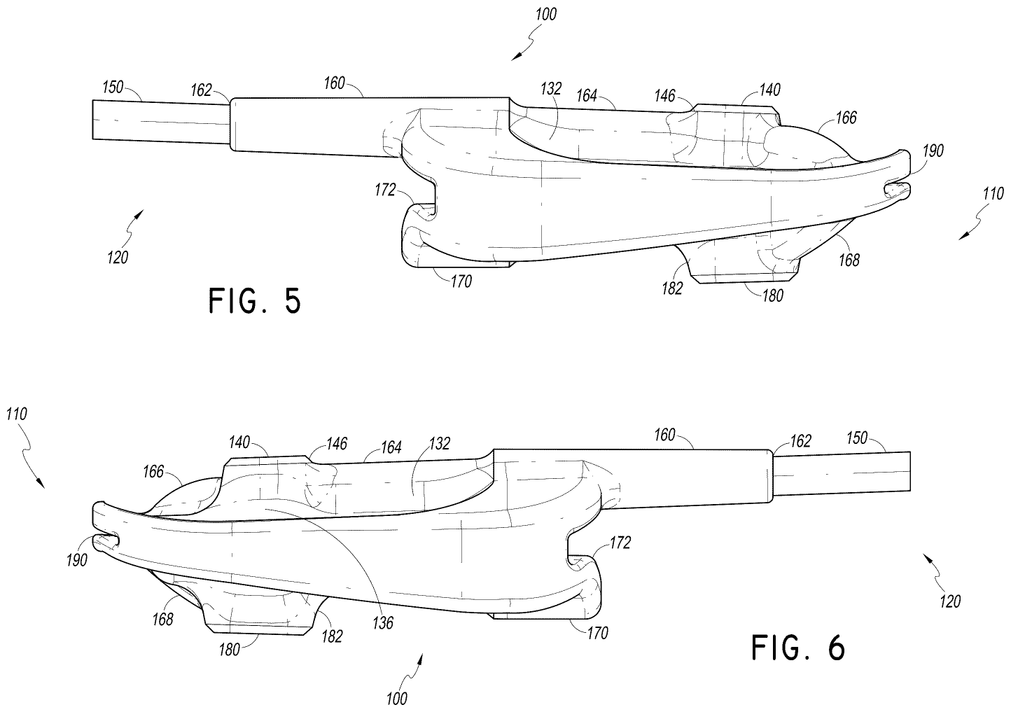

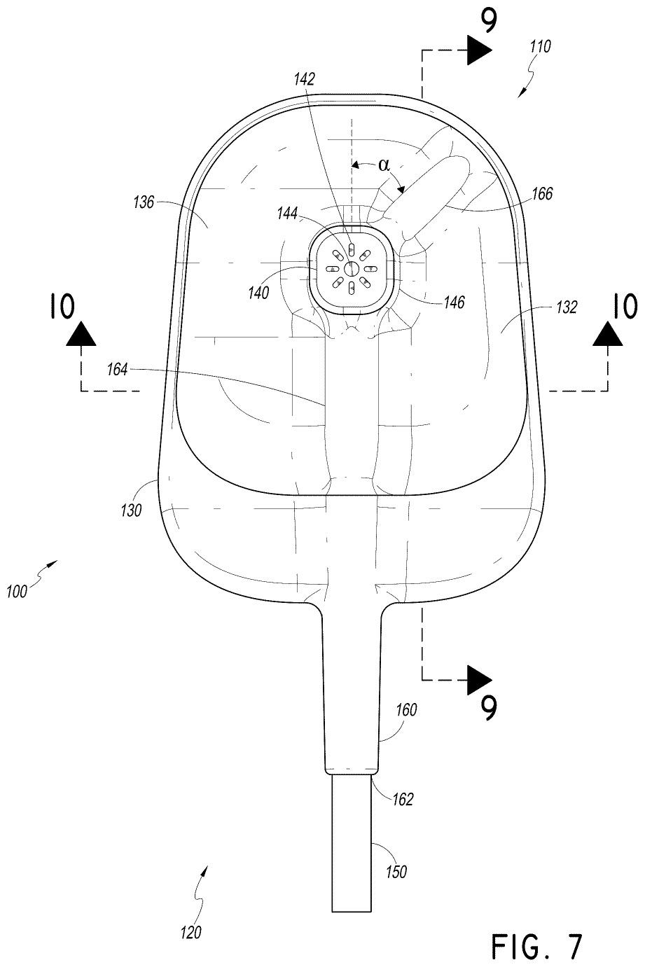

The distal end 120 of the sensor housing 130 includes a lip 170. As can be seen in FIGS. 1A and 3, the lip 170 forms the opening 172 to the sensor housing 130 which allows an individual to place his/her finger into the inside surface 17 4 of the sensor housing 130. As can be seen, the profile of the lip 170 is wider than the remainder of the sensor housing 130. In some embodiments, this allows easier placement of the sensor housing 130 on an individual's finger. In other embodiments, the lip 170 provides greater stability for the sensor 100. The thicker material of the lip 170 delivers a better grip on the portion of the finger the lip 170 is in contact with. The lip 170 also provides a light blocking barrier to prevent ambient light from entering into the sensor and causing erroneous readings. In some embodiments, because the proximal end 110 of the sensor 100 can provide a tight grip on the patient's fingertip, an equally tight grip on the finger helps provide stability if the location of that tight grip is located away from the finger tip. The lip 170 can therefore be included to provide stability. The thicker material of the lip 170 therefore facilitates the placement of the sensor 100 on a variety of finger diameters but also provides the necessary grip on the finger tip of the sensor 100 at the proximal end 110. In some embodiments, the lip 170 can also provide a spring force that grips onto the finger. In some embodiments, this allows the sensor 100 to maintain a stable grip on the finger even if the patient was to move or shake his or her hand vigorously. FIGS. 2 and 4 illustrate the slit 190 on the distal end 120 of the sensor housing 130. The slit 190 extends through the distal end 120 of the sensor housing 130 and helps the sensor housing 130 accommodate a variety of different finger sizes. In some embodiments, the slit 190 allows an individual's fingernail to protrude through the slit 190. In this way, the individual's finger can be properly positioned inside the sensor housing 130 to align with the emitter 146 and detector 182. In an embodiment, the fingernail slit 190 substantially surrounds the fingernail and acts as a light blocking barrier to prevent ambient light from entering the sensor.

In some embodiments, the material and structure of the sensor housing 130 allows the sensor housing 130 to change shape as a patient's finger is inserted. As discussed, the lip 170 of the opening 172 of the sensor housing 130 can be pulled downward to better accommodate the patient's finger as it is inserted into the body of the sensor housing 13 0. The sensor housing 13 0 surrounds the end of the patient's finger and retains the components and circuitry of the sensor 100 on the patient's finger so as to properly monitor the individual's bodily functions. Further, the material properties and shape of the sensor housing 130 help the proper positioning of the sensor 100 and provide for proper monitoring even with patient movement. The disclosed sensor housing 130 can provide proper monitoring even with a patient tapping his/her finger against a surface.

In order to monitor the individual's bodily functions as well as to connect the emitter 146 to the detector 182, the sensor housing 130 includes a wiring lumen 160 that allows wiring 150 to extend through the sensor housing 130. In some embodiments, the wiring lumen 160 extends from the sensor housing 130 and has an opening 162 at the distal end 120 of the sensor housing 130 that allows the wiring 150 to extend into. The wiring 150 extends through the wiring lumen 160 and forms a straight portion 164 along the top of the sensor housing 13 0 until it connects with the emitter 146. In some embodiments, the wiring 150 can then have an angled top portion 166 that routes the wiring 150 off center over the distal end 120 of the sensor housing 13 0. In some embodiments, the angled top portion 166 can have an angle a with a 45 degree angle from the centerline. In other embodiments, the angled top portion 166 can have an angle a that ranges from between greater than 0 degrees to less than 90 degrees. The angled top portion 166 allows the sensor housing 130 to accommodate the individual's finger and/or fingernail and to allow it to protrude from the slit 190 of the distal end 120. Further, the angled top portion 166 can prevent the wiring 150 from being stretched out and broken when the sensor 100 is turned inside out for cleaning. Because the angled top portion 166 is routed off center, the distal end 120 of the sensor housing 130 has added material 136 in order to compensate for the angled top portion 166. The angled bottom portion 168 then connects the wiring 150 to the detector 182. In some embodiments, the added material 13 6 on the proximal end 110 of the sensor 100 can also serve the function of providing the emitter 146 and/or the detector 182 with a closer and more secure fit on the fingertip.

The distal end 120 of the sensor housing 130 can accommodate the emitter 146 and the detector 182 in the top compressed portion 132 and the bottom compressed portion 134 of the sensor housing 130 respectively. As discussed above, the location of the emitter 146 and detector 182 on the compressed distal end 120 portions of the sensor housing 130 allow the components of the sensor housing 130 to be compressed against both sides of the individual's finger.

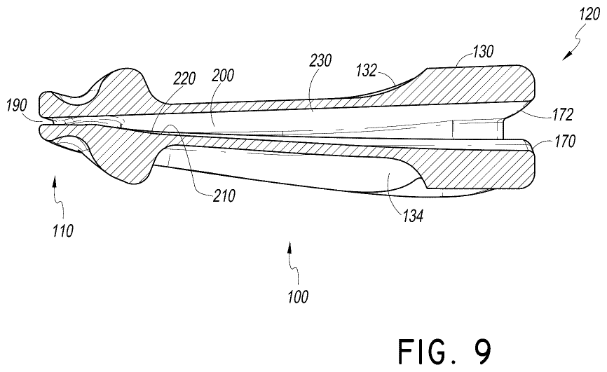

As illustrated in the figures, the emitter 146 is located near the proximal end 110 of the sensor housing 13 0 and protrudes from the surface of the top compressed portion 132. In one embodiment, the emitter 146 is centered in the distal end 120 of the sensor housing 130. The emitter 146 has a top surface 140 that is located on the surface of the sensor housing 130. FIG. 7 illustrates a top view of the sensor housing 130 and an enlarged view of the top surface 140. The top surface 140 can have portions cut out to form a design. Each of the cut-outs on the top surface 140 can be filled in with light pipe 142. The cut-outs on the top surface 140 and the light pipe 142 allows the light 144 from the emitter 146 to shine from the top surface 140. The sensor 100 can be configured to be used in environments that frequently have insufficient light (e.g. emergency situations). Therefore, the light 144 from the top surface 140 can help an individual to orient the sensor 100 in the dark. As well, in some embodiments, the sensor 100 can be configured such that the light 144 does not light up unless the sensor has been properly placed on the individual's finger. Alternatively, the light can turn off when properly placed. In such embodiments, the light 144 can help to serve as an indicator for the individual to adjust the sensor 100. The light can also provide some ambient lighting. As discussed, because the sensor 100 is frequently used in situations with insufficient light, the ambient lighting provides the additional benefit of providing a light source for emergency personnel.

The top surface 140 is located on the top compressed portion 13 2 of the sensor housing 130 and serves to protect the emitter 146. The light pipe 142 filling the cutouts in the top surface 140 help to protect the emitter 146 while still allowing light to shine from the top surface 140. The emitter 146 also has a surface that is exposed on the inside surface 174 of the sensor housing 130. In some embodiments, the emitter 146 can also serve as the light source that is captured by the detector 182 after attenuation by the body tissue.

The function of the emitter 146 described above, while shown in the context of the sensor housing 130, can be included on a variety of sensors 100. For example, the emitter in tape sensors or clip-structured sensors can be adapted such that the emitter emits light to signal to the individual that the sensor 100 is properly functioning. Alternatively, the light can turn off when properly placed. As well, the emitter in tape sensors or clip-structured sensors can be adapted to provide ambient light.

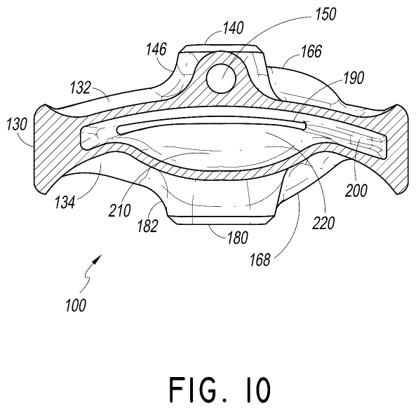

The detector 182 is located near the proximal end 110 of the sensor housing 130 on the bottom compressed portion 134. In one embodiment, the detector 182 is centered in the proximal end 110 of the sensor housing 13 0 such that it is aligned with the emitter 146. In one embodiment, the detector 182 is contained in a hard shell box 180 that protrudes from the bottom of the sensor housing 13 0 and out from the bottom compressed portion 134. FIG. 8 illustrates a bottom view of the sensor housing 130 and an enlarged view of the hard shell box 180 of the detector 182. The purpose of the hard shell box 180 is to provide for a more reliable reading from the detector 182. In some embodiments, this is accomplished by the hard shell box 180 protruding from the bottom compressed portion 134. By having the hard shell box 180 contact an outside surface instead of the outside material of the sensor housing 13 0, the sensor housing 13 0 of the sensor 100 can slide more easily on a surface and prevent catching of the sensor against a surface. In situations where the individual is moving or tapping his/her hand, if the sensor housing 13 0 of the sensor 100 were to catch on a surface, it would cause the individual's finger to slip on the inside surface 174 of the sensor housing 130 which can prevent the detector 182 from providing a reliable reading.

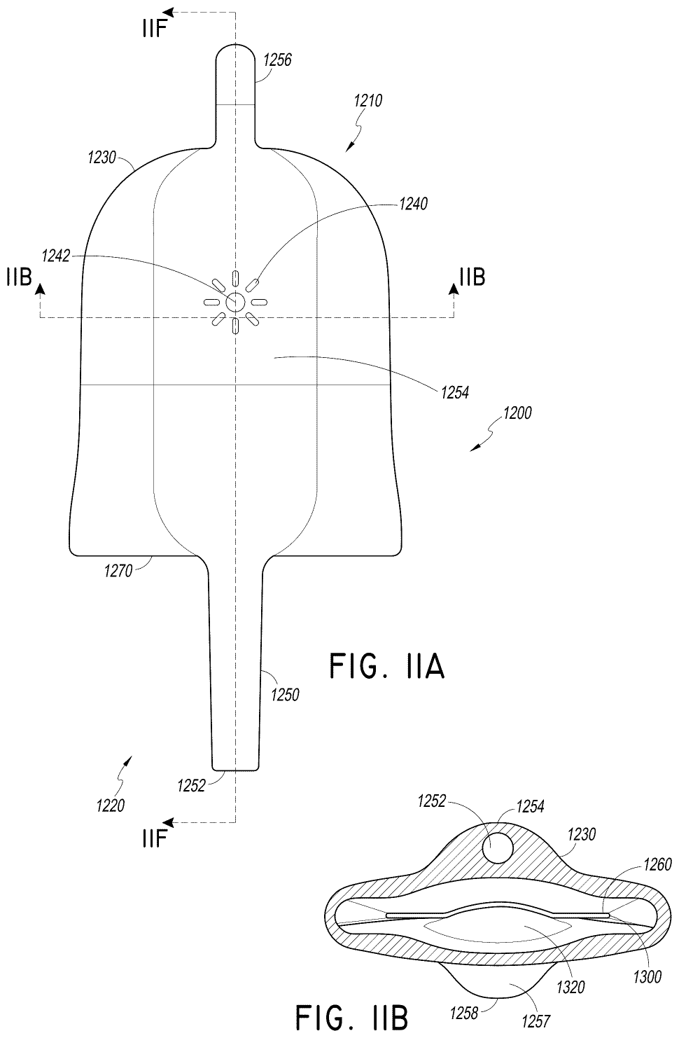

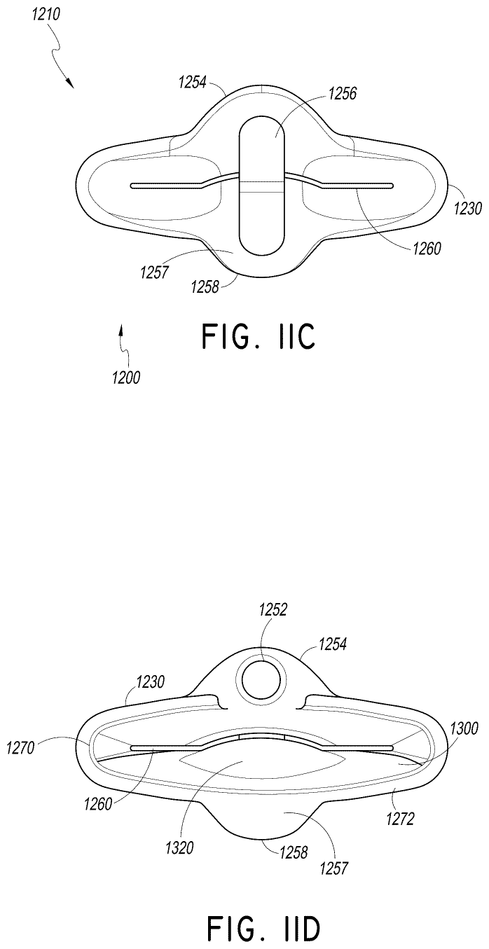

FIGS. 9-10 provide a cross-sectional view of the sensor 100. FIG. 9 illustrates an off-centered longitudinal cross-section of the sensor 100 which provides a view of the length of the inside surface 174. FIG. 10 illustrates a lateral cross-section of the sensor 100 near the proximal end 110 of the sensor 100. FIG. 9 illustrates the finger-shaped path 200 of the inside surface 17 4. The finger-shaped path 200 includes a finger lumen 23 0 and a finger-tip indentation 210 at the proximal end 220 of the finger-shaped path 200. As discussed earlier, the finger-shaped path 200 of the inside surface 174 narrows between the top compressed portion 132 and bottom compressed portion 134 which helps to capture an individual's finger between the sensor housing 130. When an individual's finger is placed in the finger-shaped path 200 of the sensor housing 130, the length of the individual's finger rests in the finger lumen 230 with the fingertip resting in the finger-tip indentation 210. As can be seen in FIG. 10, the finger-tip indentation 210 rests in between the emitter 146 and detector 182 and therefore allows the emitter 146 and detector 182 to monitor certain bodily functions. FIG. 9 also illustrates, as was discussed above, the slit 190 proximal to the fingertip indentation 210 and providing a continuous passageway to allow a fingernail to extend past the distal end 220 of the finger-tip indentation 210.

As well, as can be seen in FIG. 9, the sensor housing 130 has a thicker portion about the edges such that the center portion of the sensor housing 130 has a top compressed portion 132 and bottom compressed portion 134. In some embodiments, this thicker portion about the outer edges of the sensor 100 provides a supported frame for the sensor 100. In some embodiments, the top compressed portion 132 and bottom compressed portion 134 are composed of a thinner membrane than the surrounding portion of the sensor housing 130 and spans between the thicker portions of the sensor 100. In some embodiments, the wiring 150, emitter 146, and detector 182 is retained and floats in the thinner membrane of the top compressed portion 132 and bottom compressed portion 134 respectively. In this way, the wiring 150, emitter 146, and detector 182 are movably retained in the sensor housing 130. In some embodiments, this conformation of the sensor housing 130 provides comfort as well as accurate and stable placement of the emitter 146 and detector 182 as the sensor housing 130 can move to conform to a variety of patient finger diameters. The flexibility of the sensor 100 can therefore allow for a secure and comfortable fit of the sensor 100 about a patient's fingertip.

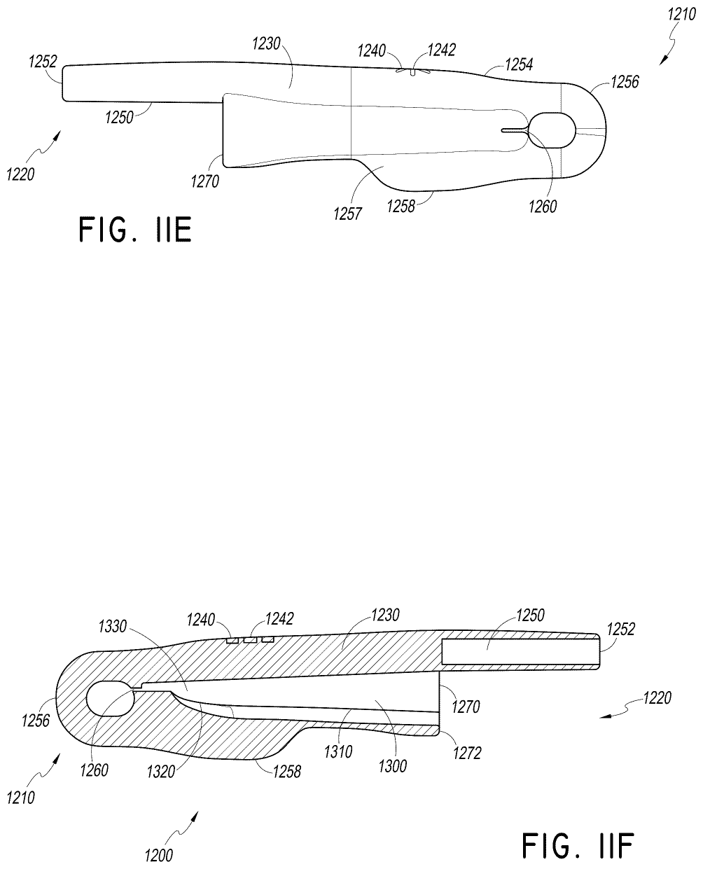

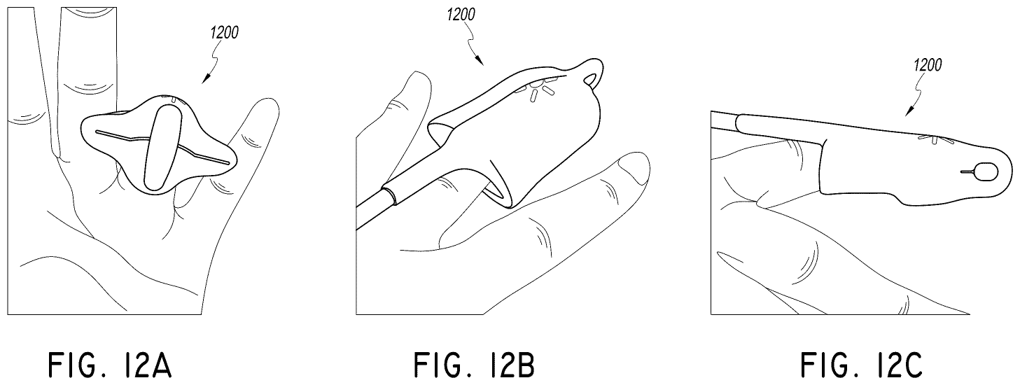

FIGS. 11A-11F and FIGS. 12A-12C provide an example of another embodiment of the sensor described above. As can be seen in this embodiment, the wiring is not contained within the sensor and protrudes from the distal end of the sensor. Unlike the wiring 150 described in the embodiment shown in FIGS. 1-10, the wiring is not routed at an angle to one side of the distal end of the sensor.

FIGS. 11A-11F illustrate the sensor 1200 with a proximal end 1210 and a distal end 1220. As described above, the sensor 1200 has a sensor housing 1230 with an opening 1270 on the distal end 1220 that allows an individual's finger to be positioned within. Like the sensor housing 130 of the sensor 100, the sensor housing 1230 retains the emitter 1242 and detector 1257 in the top and bottom portions of the sensor housing 1230 respectively. In some embodiments, the emitter 1242 and detector 1257 are placed above and below the individual's fingertip. The sensor housing 1230 also includes a wiring lumen 1250 that can house wiring (not pictured) that runs through the body of the sensor housing 1230. The sensor 1200 also includes a slit 1260 at the proximal end 1210 of the sensor housing 123 0. As discussed above, the slit 1260 sits at the distal end of the inside passageway of the sensor 1200 and allows an individual's fingernail to protrude from the distal end of the sensor 1200.

FIGS. 11A and 11E illustrate the wiring lumen 1250 through the sensor housing 1230. The wiring lumen 1250 has an opening 1252 that allows the wiring to enter the sensor housing 1230. The wiring lumen 1250 has a straight portion 1254 that connects the wiring to the emitter 1242. Unlike the wiring 150 of the sensor 100, the wiring of the sensor 1200 is not guided off-center on the sensor housing 1230. In this embodiment of the sensor 1200, because the wiring is not contained within the sensor housing 1230, the wiring does not need to be directed to either side so as to allow the opening 1270 to be placed at the proximal end 1210 of the sensor housing 1230. Instead the wiring lumen 1250 has curved portion 1256 that brings the wiring to the underside of the sensor housing 123 0 and connects the wiring from the emitter 1242 to the detector 1257.

Next, as is illustrated in FIG. 11A, in this embodiment, the surface of the sensor housing 1230 covering the emitter 1242 can have cut-outs that are filled in with light pipe 1240. The light pipe 1240 allows the light from the emitter 1242 to shine through which can, as discussed above, help an individual to orient the positioning of the sensor 1200 on the finger. In other embodiments, the light from the emitter 1242 can indicate to the individual whether the sensor 1200 is properly positioned on the finger.

FIGS. 11B and 11F provide a cross-sectional view of the sensor 1200. FIG. 11B illustrates a longitudinal cross-sectional view of the sensor 1200 which provides a view of the length of the finger-shaped path 1300. FIG. 11F illustrates a lateral cross-section of the sensor 1200 near the proximal end 1210 of the sensor 1200. FIG. 11B illustrates the finger-shaped path 1300 that includes a finger lumen 1310 and a finger-tip indentation 1320 at the proximal end 1330 of the finger-shaped path 1300. As seen in FIG. 11F, the finger-shaped path 1300 narrows towards the proximal end 1330 of the finger-shaped path 1300. When an individual's finger is placed in the finger-shaped path 1300 of the sensor housing 1230, the length of the individual's finger rests in the finger lumen 1310 with the fingertip resting in the finger-tip indentation 1320. As can be seen in FIG. 11B, the finger-tip indentation 1320 rests in between the emitter 1242 and detector 1257 and therefore allows the emitter 1242 and detector 1257 to monitor the individual's bodily functions. FIG. 11F also illustrates, as was discussed above, the slit 1260 opening proximal to the finger-tip indentation 1320 that can allow the individual's fingernail to extend past the proximal end 1330 of the finger-tip indentation 1320.

Finally, FIGS. 12A-12C provide front, top, and side views of the sensor 1200 as it is used by an individual to measure the individual's bodily functions. As can be seen, the individual's finger extends into the sensor housing 1230 from the opening 1270 on the distal end 1220 of the sensor 1200 and into the finger-shaped path 1300. The finger extends through the finger-shaped path 1300 of the sensor 1200 such that the length of the finger rests in the finger lumen 1310 and the fingertip rests in the finger-tip indentation 1320 of the proximal end 1330. At the finger-tip indentation 1320, the fingertip is between the emitter 1242 and detector 1257 that monitors certain bodily functions of the individual.

Although this disclosure has been disclosed in the context of certain preferred embodiments and examples, it will be understood by those skilled in the art that the present disclosure extends beyond the specifically disclosed embodiments to other alternative embodiments and/or uses of the disclosure and obvious modifications and equivalents thereof. In addition, while a number of variations of the disclosure have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. Accordingly, it should be understood that various features and aspects of the disclosed embodiments can be combined with or substituted for one another in order to form varying modes of the disclosed.

What is claimed is:

1. A sensor for measuring a signal indicative of at least one physiological parameter of tissue, the sensor comprising:
a sensor housing configured to be disposed about a finger of a user, the sensor housing including rubber or an elastomeric material, a distal end of the sensor housing comprising a lip forming an opening of the sensor housing to receive the finger, the sensor housing further comprising a top compressed portion and a bottom compressed portion;
an emitter contained in the top compressed portion, the emitter configured to emit light of at least two different wavelengths;
a hard shell box contained in the bottom compressed portion, the hard shell box comprising a material harder than the rubber or the elastomeric material, a portion of the hard shell box protruding from a bottom surface of the sensor housing; and
a detector contained in the hard shell box, the detector configured to detect the light after attenuation of the light by body tissue of the user.

2. The sensor of claim 1, wherein the emitter and the detector are part of an electrical circuit, wherein the electrical circuit further comprises a wire providing a connection between the emitter and the detector.

3. The sensor of claim 2, wherein the sensor housing further comprises a wire lumen extending from the sensor housing and configured to allow the wire to extend through.

4. The sensor of claim 3, wherein the wire lumen includes an opening at the distal end of the sensor housing.

5. The sensor of claim 4, wherein the wire lumen extends alongside a topside of the sensor housing.

6. The sensor of claim 5, wherein the wire extends across the topside of the sensor housing and past a proximal end of the sensor housing such that a proximal opening of the sensor housing is configured to allow a fingernail to protrude therethrough.

7. The sensor of claim 3, wherein the wire lumen and the wire has a portion that deviates from a centerline of the sensor housing at a non-zero angle.

8. The sensor of claim 7, wherein the non-zero angle is between 1 and 45 degrees.

9. The sensor of claim 2, wherein the sensor is configured to be flipped inside out without breaking the wire.

10. The sensor of claim 1, wherein a profile of the lip is wider than a remainder of the sensor housing.

11. The sensor of claim 1, wherein a top and bottom surface of the sensor housing is compressed at a proximal end of the sensor.
